(19) United States
(12) Patent Application Publication
(10) Pub. No.: US 2024/0009341 A1
(43) Pub. Date: Jan. 11, 2024

(54) DRESSING, A KIT COMPRISING A DRESSING AND A METHOD OF PREPARING A DRESSING

(71) Applicant: Chemence Medical Inc., Alpharetta, GA (US)

(72) Inventors: Stephen Hynes, Roswell, GA (US); Deog-il Kim, Marietta, GA (US)

(73) Assignee: Chemence Medical Inc., Alpharetta, GA (US)

(21) Appl. No.: 18/108,443

(22) Filed: Feb. 10, 2023

Related U.S. Application Data
(63) Continuation-in-part of application No. 17/859,164, filed on Jul. 7, 2022, now abandoned.

(51) Int. Cl. A61L 15/42; A61L 15/44; A61L 15/58; A61L 15/60

(52) U.S. Cl. CPC A61L 15/425; A61L 15/44; A61L 15/58; A61L 15/60

(57) ABSTRACT

The present invention provides a dressing for application onto a wound. The dressing comprises a porous mesh having a wound-facing side coated with an adhesive for adhering the porous mesh to the skin. The porous mesh is also configured for application of a polymerizable adhesive such that after application the polymerized adhesive and the porous mesh together form a barrier covering the wound. The dressing also includes an absorbent patch disposed on the wound-facing side of the porous mesh and arranged to overlie the wound during use.

### CROSS REFERENCE TO RELATED APPLICATIONS

This application is a continuation-in-part application of Ser. No. 17/859,164 filed Jul. 7, 2022, which is incorporated herein by reference in its entirety.

This invention relates to a dressing for application onto a wound, and a kit comprising a dressing and a method of preparing a dressing.

### BACKGROUND

Wound care dressings have a wide variety of functions and applications. One subset of wound dressings consist of a flexible, permeable mesh backed with a pressure-sensitive adhesive (PSA) which is placed over a wound (unsutured or as an adjunct to sutures). The mesh is permeable, and the PSA is pattern-coated to allow a polymerizable adhesive, typically cyanoacrylate, to penetrate the mesh and form a barrier over the mesh and wound. Such a dressing is known, for example, from US2005182443A1.

US20210212676A1 and US2018303967A1 both disclose similar dressings. US20210212676A1 additionally discloses that the mesh comprises windows for drainage of wound exudate away from the wound. A flap is provided to cover the openings and provide access for wound inspection. US2018303967A1 discloses that the mesh is adhered to the skin partially by traces of soluble pressure sensitive adhesive. After application, the soluble pressure sensitive adhesive is dissolved by wound exudate, creating a drainage channel to convey wound exudate away from the wound. Absorbent pads may be provided on a periphery of the mesh to absorb the wound exudate.

It is possible to incorporate antimicrobial agents into cyanoacrylate. However, the drawbacks to this include poor stability of the formulation and a reduced efficacy of the antimicrobial effect, as the antimicrobial agent may be trapped and unable to destroy the bacteria. Where an antimicrobial agent is provided on the mesh prior to coating with a PSA, this also has drawbacks as the impregnated mesh makes the manufacturing process more complicated, the subsequent application of the PSA reduces the potency and/or availability of the antimicrobial agent which can result in inconsistent potencies depending on how much liquid adhesive is applied to the mesh, and the antimicrobial agent may interfere with the initiation of the cyanoacrylate.

### BRIEF SUMMARY OF THE DISCLOSURE

Viewed from a first aspect, the present disclosure provides a dressing for application onto a wound. The dressing comprises: a porous mesh comprising a wound-facing side coated with an adhesive for adhering the porous mesh to the skin, the porous mesh being configured for application of a polymerizable adhesive such that after application the polymerized adhesive and porous mesh together form a barrier covering the wound. The dressing also comprises an absorbent patch disposed on the wound-facing side of the porous mesh and arranged to overlie the wound during use.

Thus, the dressing, in particular the absorbent patch, prevents contact between the polymerizable adhesive and the wound, which may reduce irritation and foreign body reactions caused by adhesive penetration at the wound. The present dressing also allows for the absorption of wound exudate from the covered wound by the absorbent patch, which can prevent the exudate from interfering with polymerization of the adhesive, thus improving the formation of the barrier. In addition, the absorbent patch may prevent the exudate from escaping from the dressing after application of the polymerizable adhesive, resulting in a longer lasting dressing and barrier.

In addition, as the polymerizable adhesive is not applied directly to the wound site, the present dressing may cause less damage to the healed tissue at the wound site during removal of the dressing.

In some cases, the absorbent patch comprises a gel-forming material. In some cases, the absorbent patch, for example the gel-forming material, may comprise any one or more of a biosynthetic material, a hydrogel, a foam, or a hydrocolloid. Biosynthetic materials can include alginate, collagen, hyaluronic acid, honey and chitosan, cellulose. The gel-forming material may be provided in a gel-forming layer of the absorbent patch. The gel-forming material may advantageously provide a more effective wound-healing environment compared to prior art dressings. In particular, the gel-forming material may maintain a moist wound environment within the barrier provided by the porous mesh and polymerized adhesive. Such a sealed, moist environment may promote wound healing by the body's own enzymes. Chitosan, cellulose and hyaluronic acid are particularly effective at providing enhanced wound healing effects.

In some examples the adhesive and/or the absorbent patch comprises a medicament. The medicament may be for any combination of: preventing colonization of the dressing, infection control, wound healing and cosmesis. The medicament may be an antimicrobial agent. The present dressing allows medicaments to be more easily incorporated into the wound contacting material or absorbent patch to aid prevention of colonization of the dressing, infection control, wound healing, cosmesis. Silver, chitosan and honey are examples of antimicrobial agents which are particularly advantageous.

In some examples the medicament is provided on a wound facing side of the absorbent patch. This may shield the medicament from the polymerizable adhesive and prevent interference between the medicament and the polymerizable adhesive, which may otherwise cause instability in the polymerizable adhesive and/or reduced effectiveness of the medicament. In some examples, the medicament is present in the adhesive in a range of between Oppm and about 5000 ppm (e.g., between 0 ppm and 5000 ppm), preferably between about 200 ppm and about 1 000 ppm (between 200 ppm and 1 000 ppm).

In some examples the absorbent patch may comprise a liner portion and an absorbent portion. The liner portion may be substantially impermeable, particularly substantially water-impermeable. The liner portion may be in contact with the porous mesh, and the absorbent portion may face the wound. The liner portion may help to prevent the polymerizable adhesive from reaching the wound during application, and may also help prevent wound exudate from reaching the mesh before, during and after application of the polymerizable adhesive. In addition, if the absorbent patch comprises a medicament, as described above, the liner portion may help to separate the polymerizable adhesive from the medicament during application of the polymerizable adhesive.

In some cases the porous mesh extends beyond an outer perimeter of the absorbent patch. The porous mesh may extend beyond the absorbent patch by at least 1 centimetre. In some cases the porous mesh may extend beyond the absorbent patch by less than 1 centimetre. In some cases, the porous mesh may circumscribe the absorbent patch. In this way, the porous mesh is adhered to the skin by the polymerizable adhesive about the entire periphery of the absorbent patch, providing a sealed wound-healing environment. In examples, the adhesive on the porous mesh may also circumscribe the absorbent patch, and may extend beyond the absorbent patch by at least 1 centimetre. In this way, the dressing can be secured to the skin by the adhesive before application of the polymerizable adhesive.

The dressing may comprise a releasable liner releasably attached to the wound facing side of the porous mesh and covering the absorbent patch. The releasable liner may comprise a central portion overlying a portion of the porous mesh and absorbent patch, and one or more peripheral portions separable from the central portion. One or both of the peripheral portions may partially overlie the absorbent patch, or may be spaced from the absorbent patch. The porous mesh may comprise an initiator for curing the polymerizable adhesive after application the polymerized adhesive to the porous mesh. The absorbent patch may comprise any of polyurethane, silicone or calcium.

There is also provided a kit for dressing a wound. The kit comprises: a dressing as described above, and a liquid polymerizable adhesive. In examples, the kit may comprise a dispenser containing the polymerizable adhesive. In examples, the dispenser further comprises a polymerization rate accelerator or modifier. In some examples the polymerizable adhesive comprises cyanoacrylate, in particular a formulation of 2-octylcyanoacrylate. In some examples, the polymerizable adhesive comprises a light cure acrylate, for example a UV-curable adhesive.

There is also provided a method of preparing a dressing for application onto a wound comprising: providing a porous mesh comprising a wound-facing side, the porous mesh being configured for application of a polymerizable adhesive such that after application the polymerized adhesive and the porous mesh together form a barrier covering the wound, at least partially coating the wound-facing side of the porous mesh with the adhesive, and applying an absorbent patch to the wound-facing side of the porous mesh, such that the absorbent patch is arranged to overlie the wound during use. At least one of the adhesive and/or the absorbent patch comprises a medicament.

The method may comprise dissolving the medicament into the adhesive prior to adhering adhesive to the porous mesh.

When the medicament is dissolved in the adhesive, it may be present in a concentration of between Oppm and about 5000 ppm (e.g., between 0 ppm and 5000 ppm), preferably between about 200 ppm and about 1 000 ppm (e.g., between 200 ppm and 1000 ppm).

### BRIEF DESCRIPTION OF THE DRAWINGS

Embodiments of the invention are further described hereinafter with reference to the accompanying drawings, in which:

FIG. 1 is a schematic representation of an exemplary dressing with a releasable liner;

FIG. 2 is a schematic illustration of the dressing with the releasable liner removed, showing an exemplary mesh and an absorbent patch;

FIG. 3 is a further illustration of the dressing, showing the relative positions of the release liner, mesh, and absorbent patch.

FIG. 4 illustrates an exploded cross-sectional view of the dressing with the release liner omitted;

FIG. 5 illustrates a polymerizable adhesive liquid being applied to the dressing; and

FIG. 6 illustrates the dressing coated with the polymerizable adhesive.

### DETAILED DESCRIPTION

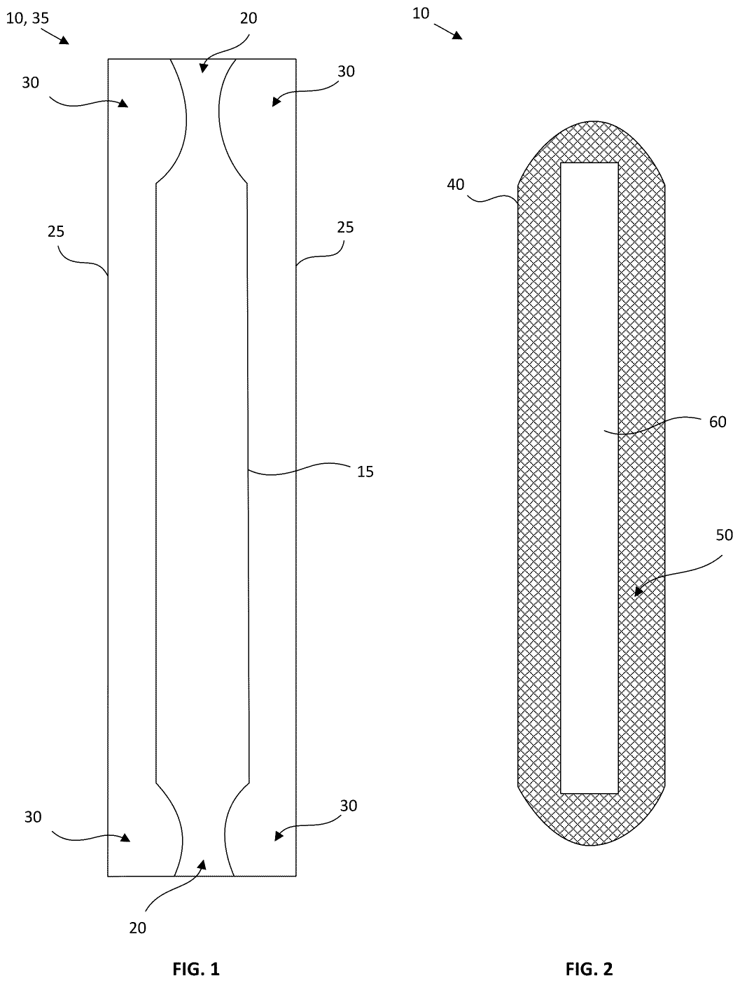

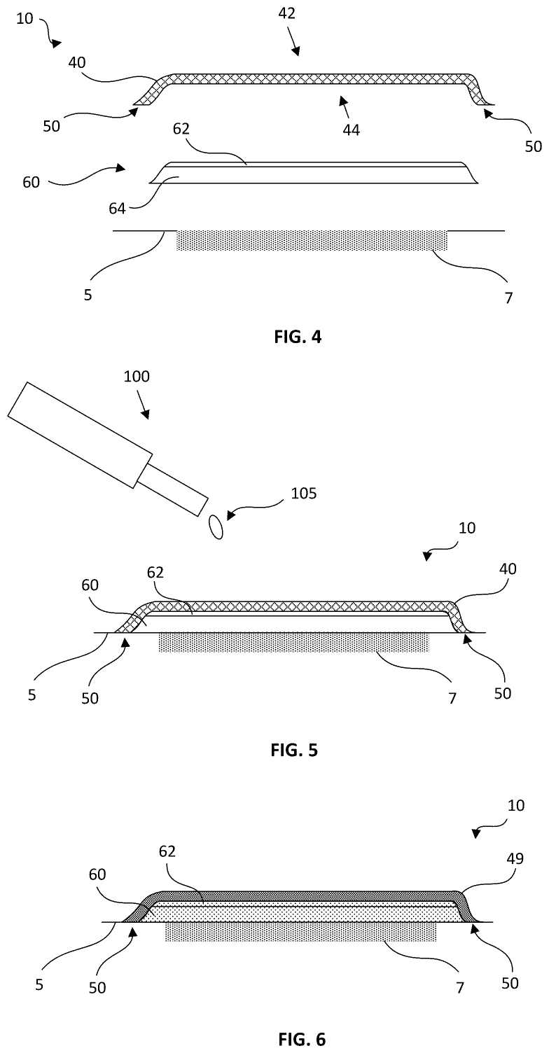

FIG. 1 is a schematic representation of an exemplary dressing 10 with a releasable liner 35. FIG. 1 shows the dressing 10 from the wound-facing side, before removal of the releasable liner 35. The releasable liner 35 is attached to a mesh 40 and absorbent patch 60 of the dressing 10, shown in FIG. 2 and described further below. The releasable liner 35 has a central portion 15 and two peripheral portions 25 which can be peeled from the mesh 40 as the dressing 10 is applied to the wound 7 (see also FIGS. 4 to 6). Tab portions 20, 30 are provided at the ends of the respective central portion 15 and the peripheral portions 25 to aid peeling. While tabs 20, 30 are shown on the ends of the central portion 15 and peripheral portions 25, it would be apparent this was not essential and that in some cases, one or both of the tabs 20, 30 could be omitted. It is also not essential to provide a central portion 15 and two separate peripheral portions 20 as illustrated. In some cases, the releasable liner 35 may be a single portion covering the mesh 40 and absorbent patch 60, or the releasable liner 35 may have more than two peripheral portions 25 . In one example, the releasable liner 35 has a central portion 15 having a tab at one end and the other end spaced from an end of the dressing 10. In this example the releasable liner 35 has a single wrap-around peripheral portion.

FIG. 2 is a schematic illustration of the dressing 10 of FIG. 1 with the releasable liner (35, see FIG. 1) removed. FIG. 2 shows a wound-facing side of the dressing 10. As shown, the dressing 10 comprises a mesh 40 and an absorbent patch 60. The illustrated mesh 40 has an adhesive 50 applied thereto for adhering the mesh 40 to the skin of a patient. The adhesive 50 is applied to a wound-facing side of the mesh 40. The adhesive is preferably a pressure-sensitive adhesive (PSA) 50 which adheres on the skin. The PSA 50 is preferably pattern-applied, with areas of the mesh 40 being coated with PSA 50 and other areas of the mesh 40 being free of PSA 50. When the dressing 10 is adhered to the patient by the PSA 50 on the mesh 40 the absorbent patch 60 overlays the wound 7 as shown in FIGS. 5 and 6. In some examples, the absorbent patch 60 may also comprise an adhesive, for example a PSA, for adhering to the patient.

The mesh 40 is porous to permit a liquid polymerizable adhesive to penetrate the mesh 40, as described further hereinafter. The mesh 40 can be formed in a number of ways, for example as a woven layer or as an interconnected lattice of fibres. The absorbent patch is absorbent to wound exudate. As explained further below, the absorbent patch 60 may comprise a liner portion and an absorbent portion. The liner portion may help to prevent the liquid polymerizable adhesive from reaching the wound, and may additionally or alternatively help prevent wound exudate from leaving the wound area during application of the dressing. As shown in FIG. 2, the mesh 40 is larger than the absorbent patch 60 and extends beyond the absorbent patch 60 around the entire periphery of the absorbent patch 60. While the mesh 40 is shown extending around the entire periphery of the absorbent patch 60, this was not essential. The absorbent patch 60 could extend to one or two edges of the mesh 40 to allow for a first dressing, similar to dressing 10 illustrated in the Figures, to be joined to a second dressing without a break in the absorbent patch 60 between the first and second dressings. The first and second dressings can be combined where the absorbent patch 60 extends to the edge of the mesh 40 to cover the wound. A continuous absorbent patch 60 from two dressings may be formed by the physician cutting the end of the first dressing, specifically the mesh 40, and partially covering the absorbent patch 60 of the first dressing with the mesh 40 of the second dressing.

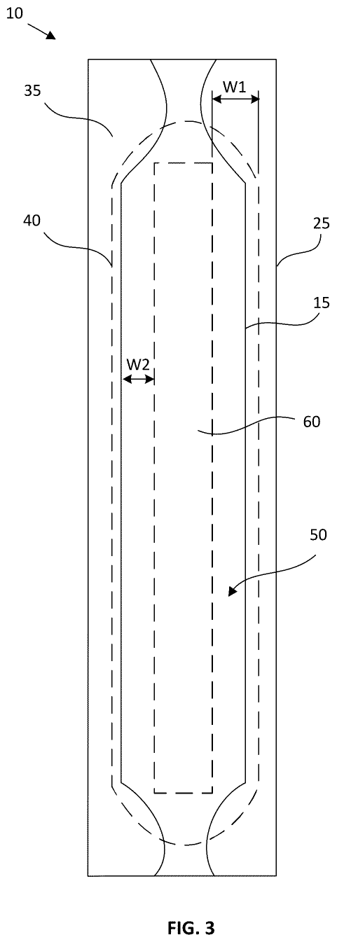

FIG. 3 shows the relative positions of the portions 15, 25 of the releasable liner 35, the mesh 40, and the absorbent patch 60. The mesh 40 and absorbent patch 60 are shown in dotted lines. As shown, the central portion 15 of the releasable liner 35 overlaps the entirety of the absorbent patch 60 and partially overlaps the mesh 40. The peripheral portions 25 partially overlap the mesh 40. In this way, the absorbent patch 60 and a portion of the mesh 40 are exposed on removal of the central portion 15, allowing the mesh 40 to be adhered to the patient with the absorbent patch 60 overlying the wound.

In particular, as shown in FIG. 3, on removal of the central portion 15 of the releasable liner 35 the part of the mesh 40 indicated by W2 is exposed, and so the PSA 50 applied to this part W2 of the mesh 40 can be used to adhere the dressing 10 at the wound site while the peripheral portions 25 remain attached to the dressing 10.

As shown in FIG. 3, a distance between the edge of the absorbent patch 60 and an edge of the mesh 40 is indicated by W1. This distance W1 is preferably at least 1 centimetre about the entire periphery of the absorbent patch 60. As explained below, the part of the mesh 40 indicated by W1 is the part that is adhered to the skin of the patient, so needs to be of sufficient width to provide a strong bond to the skin and create an effective barrier.

FIG. 4 illustrates an exploded cross-sectional view of the dressing 10 with the releasable liner (35, see FIG. 1) omitted. The thicknesses of the mesh 40 and absorbent patch 60 are exaggerated for clarity of illustration. The mesh 40 has a wound-facing side 44 and a top side 42 arranged to face externally (i.e. away from the wound 7) when the dressing is applied. The PSA 50 is applied to the wound-facing side 44. The mesh 40 provides a flexible, permeable layer which can conform to the contours of the skin 5.

The absorbent patch 60 is provided on the wound-facing side 44 of the mesh 40 and partially covers the PSA 50. The absorbent patch 60 is flexible to conform to the contours of the skin 5. The absorbent patch 60 is designed to completely cover the wound 7. For example, a wound having a length of up to 58 cm may be dressed by an absorbent patch 60 with a length of at least 60 cm. The absorbent patch 60 can be provided in a range of shapes and thicknesses as required. For example, while a rectangular absorbent patch is shown in the Figures, it would be apparent this was not essential, and the absorbent patch 60 may have an obround profile, or other shape. By providing the absorbent patch 60 on the wound-facing side of the mesh 40, exudate from the wound 7 is absorbed directly into the absorbent patch 60, substantially eliminating the risk of exudate penetrating through to the top side 42 of the mesh 40 (see FIG. 5). While an obround mesh 40 is shown in FIG. 2, it would be apparent that the mesh 40 can be provided in a range of shapes and thicknesses as 1.0 required.

The absorbent patch 60 typically comprises a material suitable for wound contact. The absorbent patch 60 comprises a material capable to absorbing wound exudate. By way of example, the absorbent patch 60 may contain a gel-forming material, for example any combination of: a hydrocolloid, an alginate, a cellulose, a foam, or a hydrogel. Gel-forming materials preferably react with the wound exudate to gradually increase the moisture level within the wound environment, encouraging moist wound healing. In some examples, the absorbent patch 60 may comprise polyurethane or silicone. For example, any of a polyurethane foam, a silicone foam, or an alginate fibrous patch or foam may provide suitable absorbent patches 60.

In examples, the absorbent patch 60 may comprise an absorbent material, for example a fibrous patch, such as cellulose fibre or cotton, for example a gauze or surgical sponge. The absorbent patch 60 may have a permeable coating facing the wound to permit absorption of exudate into the absorbent patch 60. The permeable coating on the absorbent patch 60 may allow the dressing 10 to be easily separated from the healed wound when the dressing 10 is removed.

In examples, the absorbent patch 60 comprises a liner portion 62 directed towards the mesh 40, and an absorbent portion 64 directed towards the wound 7. The absorbent portion 64 may additionally have a permeable coating facing the wound. The absorbent patch 60 may comprise an adhesive, on one or both sides, for adhering to the mesh 40 and/or to the wound 7 and skin 5. The absorbent portion 64 of the absorbent patch 60 may comprise an absorbent material, such as cellulose fibre or cotton, for example a gauze or surgical sponge.

In one specific example, the absorbent portion 64 may comprise an absorbent adhesive, for example a hydrocolloid-containing adhesive. Such an absorbent adhesive may limit break down upon saturation to assist in creating optimal skin and wound healing conditions while maintaining a high fluid handling capacity. In various examples, the absorbent patch 60 may contain an integrated hydrocolloid formulation. The hydrocolloid formulation may be provided in the absorbent portion 64 of the absorbent patch 60, or elsewhere, for example as a separate component of the absorbent patch 60.

The absorbent patch 60 may have a thickness between 100 μm and 4000 μm, preferably between about 300 μm and about 2000 μm, more preferably between about 800 μm and about 1500 μm.

The absorbent patch 60 may comprise a transfer tape, for example the MED 2191 H Transfer Tape manufactured by Avery Dennison™. The transfer tape may comprise a liner and an absorbent adhesive. The absorbent adhesive may have a high fluid handling capacity. The absorbent adhesive may include an hydrocolloid formulation. The liner of the transfer tape can be removed prior to assembly of the hydrocolloid layer to the mesh 40.

In some examples one or more medicaments, such as an antimicrobial, may also be incorporated into the absorbent patch 60 or the PSA 50. The medicament(s) may act to aid any combination of: preventing bacterial colonization of the dressing, infection control, wound healing and cosmesis. Silver is a suitable antimicrobial which may be present in the absorbent patch 60 and/or in the PSA 50. Where the antimicrobial is present in the absorbent patch 60 this may be in the form of a silver alginate foam, a polyurethane foam with silver, a silicone foam with silver, or a hydrocolloid gel with silver. Where the antimicrobial is present in the PSA 50, this can be achieved by dissolving the antimicrobial into the PSA 50 prior to coating the porous mesh 40 with any adhesive. Dissolving the antimicrobial into the PSA 50 before application of the PSA 50 to the mesh 40 advantageously reduces the complexity of the process to prepare a dressing 10. Concentrations of up to about 5000 ppm of silver, in particular SmartSilver® WC-10, have been found to be suitable for use with the presently described PSA 50. Preferably, the silver is present in concentrations between about 200ppm and about 1000 ppm in the PSA 50. It would be apparent that pure metallic silver nanoparticles in aqueous polymer solution, such as is found in ACM-5 and WC-10, is merely one example of a suitable antimicrobial agent suitable for use with the present dressing.

Table 1 below shows exemplary time-kill data for various strains of bacteria using a silver PSA 50 coated porous mesh 40 compared to a porous mesh 40 coated with regular PSA 50. Test procedures were according to The American Association of Textile Chemists and Colorists (AATCC) 100, Antibacterial Finishes on Textile Materials: Assessment of (2012) and in compliance with the ISO 13485:2016 standard.

Specified layers of sample were inoculated evenly with the challenge organism. After inoculation, samples were incubated at specified temperature for 24 hours. Immediately after inoculation, zero contact time samples were neutralized with appropriate neutralizer.

Serial dilutions were prepared and plated in duplicate using appropriate media. The 24 hour samples were processed similarly. All plates were incubated at 37±2° C. for 24-48 hours. After incubation the plates were enumerated and the percent reduction was calculated. Organisms were derived from an American Type Culture Collection (ATCC) organism, or an organism determined to be equivalent. Silver was present in a concentration of 600 ppm. For the MRSA, E. coli and P. aeruginosa samples, no Benzethonium Chloride was present on the mesh.

| | Strain/ | Silver on PSA | | No silver on PSA | |
|---|---|---|---|---|---|
| Sample | time | Uncured | Cured | Uncured | Cured |
| Staphylococcus | 0 days | 1.7 × 10\hat{ }5 | 3.5 × 10\hat{ }5 | 2.2 × 10\hat{ }5 | 3.5 × 10\hat{ }5 |
| aureus, ATCC | 7 days | <10 | <10 | 1.5 × 10\hat{ }4 | <10 |
| No. 6538 | | | | | |
| MRSA, ATCC | 0 days | 2.3 × 10\hat{ }5 | 3.9 × 10\hat{ }5 | 3.7 × 10\hat{ }5 | 3.9 × 10\hat{ }5 |
| No. 33591 | 7 days | <10 | <10 | 5.1 × 10\hat{ }4 | 5.9 × 10\hat{ }2 |
| E. coli, ATCC | 0 days | 1.3 × 10\hat{ }5 | 3.1 × 10\hat{ }5 | 3.1 × 10\hat{ }5 | 1.1 × 10\hat{ }5 |
| No. 8739 | 7 days | 4.0 × 10\hat{ }5 | <10 | 3.4 × 10\hat{ }4 | 3.4 × 10\hat{ }5 |
| Pseudomonas | 0 days | 3.3 × 10\hat{ }5 | 3.2 × 10\hat{ }5 | 3.2 × 10\hat{ }5 | 4.0 × 10\hat{ }5 |
| aeruginosa, | 7 days | <10 | <10 | <10 | <10 |
| ATCC 9027 | | | | | |

As can be seen from Table 1, the microbial count was significantly reduced by the presence of silver in the mesh 40 before treatment with cyanoacrylate and by silver or cyanoacrylate in the cured sample. For MRSA without silver there was much less reduction for both the cyanoacrylate coated sample and the mesh alone. For E. coli the combination of silver and cyanoacrylate gave a synergistic effect which was not present in the sample without silver or with just cyanoacrylate alone. Table 1 supports the effect that silver combined with the PSA 50 increases the range and breath of microbial control for the porous mesh 40 alone and when covered with cyanoacrylate. The adhesion strength of silver PSA 50 coated mesh 40 was found to be comparable to regular PSA 50 coated mesh 40. Thus the present concentrations of silver in PSA 50 are able to reduce microbial count without compromising on the adhesion strength of the dressing 10.

One way of forming an absorbent patch 60 with a medicament is to attach silver ions onto fibres making up the absorbent patch 60. For example, calcium alginate fibres can be treated with one or more aqueous solutions of silver nitrate. The aqueous solutions allow for the silver ions in solution to be exchanged for calcium ions in the fibre which forms calcium alginate fibres containing silver ions. It would be apparent that other medicaments could be attached in a similar process. It would also be apparent that while an alginate patch is described, other patches or pads containing a medicament could be used, such as a polyurethane foam, a silicone foam or a hydrocolloid gel as described herein. Where the absorbent patch 60 is to contain a hydrocolloid containing silver, this can be achieved by adding a silver compound such as silver sulfadiazine to the hydrocolloid material before film casting.

As the polymerizable liquid 105 (see FIG. 5) applied to the dressing does not penetrate through the absorbent patch 60, potentially mixing with the medicament, there is improved stability of the polymerizable adhesive and medicaments. That is, the medicaments do not interfere with the polymerization of the polymerizable adhesive, and the polymerizable adhesive does not interfere with the medicaments.

FIG. 5 illustrates the dressing 10 as applied to a wound 7. The thicknesses of the mesh 40 and absorbent patch 60 are exaggerated for clarity of illustration.

Referring to FIGS. 3 and 5, the dressing 10 is applied to the wound 7 by first removing the central portion 15 of the release liner 35 to expose the absorbent patch 60 and a part of the mesh 40. The dressing 10 is then positioned over the wound 7, with the PSA 50 on the exposed part of the mesh 40 acting to hold the edges of the wound 7 in approximation. The peripheral portions 25 of the release liner 35 allow the dressing 10 to be handled easily while the dressing 10 is positioned over the wound 7. Subsequently, the peripheral portions 25 can be removed and the remainder of the mesh 40 can be adhered to the skin 5 as shown in FIG. 5.

As illustrated in FIG. 5, once the dressing 10 has been applied the absorbent patch 60 covers the wound 7 and the mesh 40 is adhered to the skin 5 either side of the wound 7 by the PSA 50. The PSA 50 on the mesh 40 acts to secure the dressing 10 to the patient and to approximate the wound 7 (i.e., to hold the edges of the wound 7 together or in close approximation). In some examples the absorbent patch 60 also comprises an adhesive (e.g., a PSA) that serves the same purpose.

Once the dressing 10 has been positioned and adhered to the skin 5 a liquid polymerizable adhesive 105 is applied and cured to form a barrier over and around the wound, as described above. After the dressing 10 has been adhered to the skin 5 and before the polymerizable adhesive 105 has been applied and cured, the absorbent patch 60 acts to absorb wound exudate and prevent the wound exudate from penetrating the mesh 40.

Accordingly, the absorbent patch 60 reduces mixing of the polymerizable adhesive 105 and wound exudate.

FIG. 5 also illustrates a polymerizable adhesive 105, for example cyanoacrylate, being applied to the dressing 10 from a dispenser 100. The polymerizable adhesive 105 is in a liquid form when applied, and polymerizes (cures) after application. A 2-octylcyanoacrylate formulation has been found particularly suitable for use with the present dressing. The dispenser 100 may be provided with the dressing 10 in a sterile package, for example as a kit for dressing a wound 7. In use, the polymerizable adhesive 105 is applied over some or all of the top side 42 of the mesh 40 to impregnate the mesh 40. Preferably, polymerizable adhesive 105 is applied evenly over all of the mesh 40. While the described dispenser 100 includes an initiator for triggering the curing process, it would be apparent this was not essential. In some cases there may not be an initiator, and in some cases the initiator can be part of the mesh 40, for example impregnated in the mesh 40. Preferably, the liquid polymerizable adhesive 105 does not penetrate through the absorbent patch 60 to contact the wound 7. For example, the liner portion 62 of the absorbent patch 60 prevents the polymerizable adhesive 105 from penetrating to the wound 7. The mesh 40 and/or the absorbent patch 60 may be configured in a suitable manner to limit the penetration of the liquid polymerizable adhesive 105. The dispenser 100 may include a polymerisation rate accelerator or modifier that is mixed or combined with the polymerizable adhesive 105 as it is dispensed. Therefore, the polymerizable adhesive will polymerize (cure) in a short period of time after application to the mesh 40. In some cases, the liner portion 62 can be omitted entirely from the dressing 10 and the absorbent patch 60 alone is sufficient to prevent the liquid polymerizable adhesive 105 from penetrating the wound 7. Where the liner portion 62 is present this may reduce (or entirely prevent) the liquid polymerizable adhesive from penetrating the wound 7, and/or prevent movement of wound exudate from the absorbent patch 60 into the mesh 40 overlying the absorbent patch 60.

FIG. 6 illustrates the dressing 10 after application of the polymerizable adhesive 105. The polymerizable adhesive 105 is designed to polymerise and cure after being applied to the mesh 40 to form a barrier 49. Once the polymerizable adhesive 105 has cured, the mesh 40, which is now impregnated with polymerizable adhesive 105, forms a barrier 49 to outside bacterial penetration to the wound 7. The cured polymerizable adhesive 105 also reinforces the dressing, providing a stronger dressing compared to prior art wound dressings. The polymerizable adhesive 105 also helps to anchor the dressing to the skin 5 by penetrating pores within the mesh 40 and adhering to the skin 5. In particular, the polymerizable adhesive 105 penetrates areas of the mesh 40 that do not have the PSA 50 applied and bonds to the skin 5.

By providing an absorbent patch 60 between the mesh 40 and the wound 7, the present dressing 10 prevents direct contact between the polymerizable adhesive 105 and the wound 7, and thereby reduces potential irritation and foreign body reactions caused by the polymerizable adhesive 105 contacting the broken skin 5 of the wound 7. In addition, as the polymerizable adhesive 105 adheres to the skin only where the mesh 40 is in contact with the skin 5 (i.e., around the periphery of the absorbent patch 60), the healed tissue at the wound site is less damaged during removal of the dressing 10. Meanwhile, the barrier 49 extends across the entire mesh 40 and therefore the dressing 10 provides a sealed area enclosing the wound 7. In examples in which the absorbent patch contains a gel-forming material, the combination of the sealed area enclosing the wound 7 and a gel-forming material provided in the absorbent patch 60 creates an improved wound-healing environment.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

1. A dressing for application onto a wound, the dressing comprising:
a porous mesh comprising a wound-facing side coated with an adhesive for adhering the porous mesh to the skin, the porous mesh being configured for application of a polymerizable adhesive such that after application the polymerized adhesive and the porous mesh together form a barrier covering the wound; and,
an absorbent patch disposed on the wound-facing side of the porous mesh and arranged to overlie the wound during use.

2. The dressing of claim 1, wherein the absorbent patch comprises a gel-forming material.

3. The dressing of claim 1, wherein the absorbent patch comprises any of a biosynthetic material, a hydrogel, a foam, or a hydrocolloid.

4. The dressing of claim 1, wherein the porous mesh or the absorbent patch comprises a medicament.

5. The dressing of claim 4, wherein the medicament is for any combination of: preventing colonization of the dressing, infection control, wound healing and cosmesis.

6. The dressing of claim 4, wherein the medicament is present in the adhesive in a range of between 0 ppm and about 5000 ppm, preferably between about 200 ppm and about 1000 ppm.

7. The dressing of claim 1, wherein the absorbent patch comprises a liner portion and an absorbent portion, and wherein the liner portion is directed towards the porous mesh such that the absorbent portion faces the wound after application.

8. The dressing of claim 7, wherein the liner portion is substantially impermeable.

9. The dressing of claim 1, wherein the porous mesh extends beyond an outer perimeter of the absorbent patch.

10. The dressing of claim 9, wherein the porous mesh circumscribes the absorbent patch.

11. The dressing of claim 10, wherein the porous mesh extends beyond the outer perimeter of the absorbent patch by at least 1 cm.

12. The dressing according to claim 1, comprising a releasable liner attached to the wound-facing side of the porous mesh and covering the absorbent patch.

13. The dressing according to claim 12, wherein the releasable liner comprises a central portion overlying the absorbent patch and at least a portion of the porous mesh, and at least one peripheral portion separable from the central portion.

14. The dressing according to claim 1, wherein the absorbent patch comprises any of polyurethane, silicone, or calcium.

15. A kit for dressing a wound, the kit comprising:
a dressing according to claim 1, and
a liquid polymerizable adhesive for application to the dressing.

16. A kit according to claim 15, wherein the liquid polymerizable adhesive comprises cyanoacrylate.

17. A method of preparing a dressing for application onto a wound comprising:
providing a porous mesh comprising a wound-facing side, the porous mesh being configured for application of a polymerizable adhesive such that after application the polymerized adhesive and the porous mesh together form a barrier covering the wound,
at least partially coating the wound-facing side of the porous mesh with the adhesive, and
applying an absorbent patch to the wound-facing side of the porous mesh, such that, the absorbent patch is arranged to overlie the wound during use,
wherein at least one of the adhesive and/or the absorbent patch comprises a medicament.

18. The method of claim 17 comprising dissolving the medicament into the adhesive prior to adhering adhesive to the porous mesh.

19. The method of claim 18, wherein the medicament is dissolved in the adhesive at a concentration of between 0 ppm and about 5000 ppm, preferably between about 200 ppm and about 1000 ppm.
